(19) United States
(12) Patent Application Publication
(10) Pub. No.: US 2012/0310324 A1
(43) Pub. Date: Dec. 6, 2012

(54) TREATMENT OF A MAIN BODY LUMEN IN THE VICINITY OF A BRANCHING BODY LUMEN

(75) Inventors: Raphael Benary, Tel Aviv (IL); Alon Shalev, Ra'anana (IL)

(73) Assignee: ENDOSPAN LTD., Herzilyia Pituach (IL)

(21) Appl. No.: 13/505,996

(22) PCT Filed: Nov. 4, 2010
(86) PCT No.: PCT/IL10/00917

§ 371 Date: Aug. 15, 2012

Related U.S. Application Data
(60) Provisional application No. 61/257,856, filed on Nov. 4, 2009.

(51) Int. Cl. A61F 2/84; A61F 2/94

(52) U.S. Cl. 623/1.12; 623/1.35

(57) ABSTRACT

An endovascular prosthesis (100) comprises a structural member (131), which defines, when the prosthesis (100) assumes an expanded state, a substantially tubular structure (111), and two wings (107, 108), which are coupled to a proximal end (118) of the tubular structure (111). If the wings (107, 108) are placed within and in contact with a right circular cylinder (102), which has a diameter of between 2.5 and 3 cm, such that a distal end (119) of the tubular structure (111) is outside the cylinder (102): (a) an axis (116) of the tubular structure (111) defines an angle of between 75 and 90 degrees with an axis (106) of the cylinder (102), (b) the wings (107, 108) at least partially occupy respective arcs (103A, 103B) of the cylinder (102), at least one of which arcs (103A, 103B) has an angle of no more than 180 degrees, and (c) the wings (107, 108) have respective greatest axial lengths (104) along the cylinder axis (106), at least one of which is at least 1.5 times a diameter (122) of the tubular structure (111).

### CROSS-REFERENCE TO RELATED APPLICATIONS

The present patent application claims priority from U.S. Provisional Application 61/257,856, filed Nov. 4, 2009, entitled, “Method and apparatus for treatment of a main body lumen in the vicinity of a branching auxiliary lumen,” which is incorporated herein by reference.

### FIELD OF THE APPLICATION

This present application relates generally to prostheses and surgical methods, and specifically to tubular prostheses, including endovascular grafts and stent-grafts, and surgical techniques for using the prostheses to maintain patency of body passages such as blood vessels, and treating aneurysms.

### BACKGROUND OF THE APPLICATION

Endovascular prostheses are sometimes used to treat aortic aneurysms. Such treatment includes implanting a stent or stent-graft within the diseased vessel to bypass the anomaly. An aneurysm is a sac formed by the dilation of the wall of the artery. Aneurysms may be congenital, but are usually caused by disease or, occasionally, by trauma. Aortic aneurysms which commonly form between the renal arteries and the iliac arteries are referred to as abdominal aortic aneurysms (“AAAs”). Other aneurysms occur in the aorta, such as thoracic aortic aneurysms (“TAAs”) and aortic uni-iliac (“AUI”) aneurysms.

PCT Publication WO 2008/107885 to Shalev et al., and US Patent Application Publication 2010/0063575 to Shalev et al. in the US national stage thereof, which are incorporated herein by reference, describe a multiple-component expandable endoluminal system for treating a lesion at a bifurcation, including a self expandable tubular root member having a side-looking engagement aperture, and a self expandable tubular trunk member comprising a substantially blood impervious polymeric liner secured therealong. Both have a radially-compressed state adapted for percutaneous intraluminal delivery and a radially-expanded state adapted for endoluminal support.

The following references may be of interest:

U.S. Pat. No. 4,938,740

U.S. Pat. No. 5,824,040 to Cox et al.

U.S. Pat. No. 7,044,962 to Elliott

US Patent Application Publication 2006/0229709 to Morris et al.

US Patent Application Publication 2006/0241740 to Vardi et al.

US Patent Application Publication 2008/0109066 to Quinn

### SUMMARY OF APPLICATIONS

Some applications of the present invention provide an endovascular prosthesis, which is configured to be positioned at a branch between a main body lumen and a branching body lumen, such as a main blood vessel and a branching blood vessel. For example, the main body lumen may be an aorta, and the branching body lumen may be a renal artery. The prosthesis comprises a structural member and, optionally, a fluid flow guide. The prosthesis is configured to initially be positioned in a tubular delivery shaft in a compressed state, and to assume an expanded state upon being deployed from the tubular delivery shaft. When the prosthesis assumes its expanded state, respective portions of the structural member are shaped so as to define (a) a main portion, which is configured to be positioned in the main body lumen, and (b) a branching portion that comprises a substantially tubular structure, which is configured to be positioned in the branching body lumen.

For some applications, the main portion comprises two wings, which are coupled to a proximal end of the tubular structure at generally opposite sides of the proximal end. The wings are placed within and in contact with the main body lumen, such that a distal end of the tubular structure is positioned outside the main body lumen in the branching body lumen.

In some applications of the present invention, a kit is provided that comprises two or more of the prostheses, and a stent-graft that is configured to be positioned in the main body lumen. For some applications, the elements of the kit are deployed by first deploying and positioning a first one of the prostheses in one of the renal arteries, then deploying and positioning a second one of the prostheses in the other of the renal arteries. Subsequently, the stent-graft is deployed within the wings of the two prostheses. Radial expansion of the stent-graft within the wings holds the wings against the wall of the aorta, thereby further securing the prostheses in place in the aorta.

Because the prostheses are separately deployed, each can be properly positioned in one of the renal arteries, even though the renal arteries generally branch from the aorta at different respective axial positions along the aorta. In contrast, if the stent-graft itself were to comprise branching tubular structures, it would often be difficult to insert these tubular structures into the renal arteries, particularly since the renal arteries having differing axial positions in different patients. In addition, it could be necessary to use a plurality of guidewires, which would increase the crossing profile of the deployment tool.

For some applications, the stent-graft comprises a stent-graft structural member and a blood-impervious stent-graft fluid flow guide attached to the stent-graft structural member. For some applications, the fluid flow guide is shaped so as to define an axial discontinuation around at least a portion of a circumference of the stent-graft, such entirely around the circumference. The axial discontinuation typically axially overlaps with the lengths of the endovascular prostheses, in order to allow blood flow into the prostheses and the branching blood vessels.

For some applications, the two prostheses are sized to circumferentially overlap with each other, typically at two sites. This overlap serves to provide a fluid seal, thereby defining a fluid flow path through a tubular wall, effectively created by the four wings of the two prostheses. For some applications, at least a portion of the stent-graft fluid flow guide axially overlaps with the wings, thereby providing a fluid flow path between the wings and the portion of the fluid flow guide.

In order to provide a consistent overlap and fluid-tight seal between the fluid flow guide of the stent-graft and the fluid flow guides of the prostheses, it is generally desirable to position the prostheses such that the proximal (e.g., caudal) ends of the two prostheses are axially aligned with each other, and/or the distal (e.g., rostral) ends of the two prostheses are axially aligned with each other. In order to facilitate such alignment, for some applications the kit includes a plurality of prostheses in which the tubular structure joins the wings at varying axial positions. The surgeon selects two of the prostheses with appropriately-positioned tubular structures, to provide the desired axial alignment of the proximal and/or distal ends.

Typically, when the prosthesis is positioned at the branch of the main body lumen and the branching body lumen:
a central longitudinal axis of the tubular structure defines an angle of between 75 and 90 degrees with a central longitudinal axis of the main body lumen;
the wings at least partially occupy respective arcs of the main body lumen, at least one of which arcs has an angle of no more than 180 degrees; and
the wings have respective greatest axial lengths, at least one of which is at least 1.5 times a diameter of the tubular structure.

For some applications, the wings are shaped so as to define at least one gap between the wings (e.g., exactly two gaps) near the proximal end of the tubular structure. Alternatively or additionally, for some applications, the wings are not fixed to each other at any points farther than 2 mm from the proximal end of the tubular structure, when the prosthesis assumes the expanded state and if the wings are placed within and in contact with the main body lumen.

For some applications, the prosthesis is configured to be positioned in the delivery shaft in its compressed state such that:
the tube axis coincides with a central longitudinal axis of the delivery shaft;
the tubular structure is radially compressed around the tube axis;
the wings curve around the axis of the delivery shaft and subtend respective arcs of the delivery shaft; the wings may thus together define at least a portion of, such as all of, a generally tubular shape;
the wings are aligned alongside each other (such as generally parallel to each other), and (whether or not the wings overlap) define two slits between the wings, which typically extend along the entire length of the compressed prosthesis other than the tubular structure; and/or
the tubular structure and wings together define at least a portion of, such as all of, a generally tubular shape.

There is therefore provided, in accordance with an application of the present invention, apparatus for use with a tubular delivery shaft, the apparatus including an endovascular prosthesis, which is configured to initially be positioned in the delivery shaft in a compressed state, and to assume an expanded state upon being deployed from the delivery shaft, and which includes a structural member, respective portions of which are shaped so as to define, when the prosthesis assumes the expanded state:

a substantially tubular structure, having proximal and distal ends, a diameter, and a central longitudinal tube axis, and

two wings, which are coupled to the proximal end of the tubular structure at generally opposite sides of the proximal end, such that, if the wings are placed within and in contact with a right circular cylinder, which has a diameter of between 2.5 and 3 cm, such that the distal end of the tubular structure is outside the cylinder:
the tube axis defines an angle of between 75 and 90 degrees with a central longitudinal axis of the cylinder,
the wings at least partially occupy respective arcs of the cylinder, at least one of which arcs has an angle of no more than 180 degrees, and
the wings have respective greatest axial lengths along the cylinder axis, at least one of which is at least 1.5 times the diameter of the tubular structure.

For some applications, the angle of the at least one of the arcs is between 30 and 170 degrees, such as between 95 and 170 degrees. Alternatively or additionally, for some applications, each of the arcs has an angle that is no more than 180 degrees. Alternatively or additionally, for some applications, a sum of the angles of the arcs of the two wings is less than 360 degrees, such as between 90 and 270 degrees.

For some applications, each of the greatest axial lengths is at least 1.5 times the diameter of the tubular structure.

For some applications, the two wings are not fixed to each other at any points farther than 2 mm from the proximal end of the tubular structure, when the prosthesis assumes the expanded state and if the wings are placed within and in contact with the cylinder.

For some applications, the two wings are shaped so as to define at least one gap between the wings, at least a portion of which gap is within 1 cm of the proximal end of the tubular structure, when the prosthesis assumes the expanded state and if the wings are placed within and in contact with the cylinder. For some applications, the two wings are shaped so as to define exactly two gaps between the wings, at least respective portions of which are within 1 cm of the proximal end of the tubular structure.

For some applications, the two wings are fixed to each other at exactly zero, exactly one, or exactly two points. For some applications, when the prosthesis assumes the expanded state and if the wings are placed within and in contact with the cylinder, each of the wings extends' axially along the cylinder beyond the proximal end of the' tubular structure in a first axial direction and a second axial direction opposite the first direction, such that the wings define a first gap between the wings in the first axial direction, and a second gap between the wings in the second axial direction.

For some applications, the prosthesis is configured such that if the wings are placed within the cylinder, the wings contact the cylinder even if no radial forces are applied to the wings.

For some applications, the prosthesis is configured such that if the wings are placed within the cylinder, the wings contact the cylinder only if one or more forces are applied to the wings in one or more radially-outward directions. For some applications, the apparatus further includes a generally tubular self-expanding stent-graft, which is configured and sized to apply the one or more forces to the wings upon expansion of the stent-graft.

For some applications, the prosthesis is configured such that if the wings are placed within the cylinder, the wings contact the cylinder only if one or more forces are applied radially inwardly on the wings by the cylinder.

For some applications, respective tube-coupling portions of the wings are coupled to the proximal end of the tubular structure; the wings are shaped so as to define respective end portions farthest from the tube-coupling portions, when the prosthesis assumes the expanded state and if the wings are placed within and in contact with the cylinder; and the end portions are not fixed to each other.

For some applications, at least one of the wings extends axially along the cylinder beyond the proximal end of the tubular structure in a first axial direction and a second axial direction opposite the first direction, when the prosthesis assumes the expanded state and if the wings are placed within and in contact with the cylinder. For some applications, the wings together define a main portion of the prosthesis, and the tubular structure joins the wings at a junction such that a center of the junction is positioned within 5% of an axial length of the main portion from an axial center of the main portion. For some applications, the wings together define a main portion of the prosthesis, and the tubular structure joins the wings at a junction such that a center of the junction is positioned greater than 10% of an axial length of the main portion from an axial center of the main portion.

For some applications, the prosthesis is configured to be positioned in the delivery shaft in the compressed state such that the tube axis of the tubular structure coincides with a central longitudinal axis of the delivery shaft, and the wings curve around the axis of the delivery shaft and subtend respective arcs of the delivery shaft. For some applications, the two wings together define at least a portion of a generally tubular shape, when the prosthesis is positioned within the delivery shaft. For some applications, the two wings and the tubular structure together define at least a portion of a generally tubular shape, when the prosthesis is positioned within the delivery shaft. For some applications, the two wings are aligned alongside each when the prosthesis is positioned within the delivery shaft. For some applications, the prosthesis is configured to be positioned in the delivery shaft in the compressed state such that the subtended arcs of the delivery shaft do not overlap each other. For some applications, a sum of the non-overlapping subtended arcs of the delivery shaft is at least 350 degrees. For some applications, the prosthesis is configured to be positioned in the delivery shaft in the compressed state such that the subtended arcs of the delivery shaft overlap each other.

For some applications, the prosthesis further includes a blood-impervious fluid flow guide, which is attached to at least a portion of the structural member. For some applications, the fluid flow guide is attached to the structural member such that the fluid flow guide entirely covers both of the wings, such that the fluid flow guide creates a blood-impervious continuum together with the wings. For some applications, the fluid flow guide is attached to the structural member such that the fluid flow guide only partially covers each of the wings. For some applications, the fluid flow guide is attached to the structural member such that the fluid flow guide covers at least a portion of the tubular structure, which portion extends from the proximal end of the tubular structure toward the distal end of the tubular structure.

For some applications, the tubular structure has a diameter of between 3 and 12 nun, when the prosthesis assumes the expanded state. For some applications, the tubular structure has an axial length of between 1 and 5 cm, when the prosthesis assumes the expanded state.

For some applications, the structural member includes a super-elastic alloy, such as Nitinol. For some applications, the structural member includes a plurality of interconnected structural stent elements.

For some applications, respective lengths of the arcs occupied by the wings are within 10% of each other, when the prosthesis assumes the expanded state and if the wings are placed within and in contact with the cylinder.

For any of the applications described above, the apparatus may further including the delivery shaft. For some applications, the prosthesis is initially positioned at a distal end of the delivery shaft in the compressed state. For some applications, the prosthesis is initially positioned such that the prosthesis is entirely within the delivery shaft. For some applications, the delivery shaft is configured to slidably release the prosthesis therefrom. For some applications, the delivery shaft further includes an elongated inner shaft, which is positioned within the delivery shaft, and which is configured to prevent movement of the prosthesis in a distal direction within the delivery shaft. For some applications, the inner shaft is shaped so as to define an inner lumen, which is configured to allow insertion of a guidewire therethrough.

For any of the applications described above, the prosthesis may be configured to be positioned at a branch between a main body lumen and a branching body lumen, such that the tubular structure is positioned within the branching body lumen, and the wings are positioned within the main body lumen. For some applications, the main body lumen is an aorta, and the branching body lumen is a renal artery, and the prosthesis is configured to be positioned such that the tubular structure is positioned within the renal artery, and the wings are positioned within the aorta.

For any of the applications described above, the prosthesis is one of plurality of prostheses, and the apparatus includes a kit, which includes two or more of the prostheses. For some applications, the kit includes at least first and second ones of the prostheses; the wings of the first prosthesis together define a main portion of the first prosthesis, and the tubular structure of the first prosthesis joins the wings at a junction such that a center of the junction is positioned within 5% of an axial length of the main portion from an axial center of the main portion; and the wings of the second prosthesis together define a main portion of the second prosthesis, and the tubular structure of the second prosthesis joins the wings at a junction such that a center of the junction is positioned greater than 10% of the axial length from the axial center of the main portion.

For some applications, the kit includes exactly two of the prostheses.

For some applications, the kit further includes a generally tubular self-expanding stent-graft. For some applications, no portion of the stent-graft is fixed to any of the prostheses. For some applications, the stent-graft includes a stent-graft structural member and a blood-impervious stent-graft fluid flow guide attached to the stent-graft structural member, which fluid flow guide is shaped so as to define an axial discontinuation around at least a portion of a circumference of the stent-graft. For some applications, the stent-graft fluid flow guide is shaped so as to define the axial discontinuation entirely around the circumference of the stent-graft. For some applications, the axial discontinuation has a discontinuation length equal to between 50% and 85% of each of the shortest axial lengths of the wings, or between 60% and 70% of each of the shortest axial lengths of the wings.

There is further provided, in accordance with an application of the present invention, a method including:

providing an endovascular prosthesis, which is configured to assume a compressed state and an expanded state, and which includes a structural member, respective portions of which are shaped so as to define, when the prosthesis assumes the expanded state: (a) a substantially tubular structure, having proximal and distal ends, a diameter, and a central longitudinal tube axis, and (b) two wings, which are coupled to the proximal end of the tubular structure at generally opposite sides of the proximal end;

transvascularly introducing the prosthesis into a main body lumen of a human subject, while the prosthesis is positioned in a tubular delivery shaft in the compressed state; and

deploying the prosthesis from the delivery shaft at a branch between the main body lumen and a branching body lumen, such that (a) the tubular structure is positioned in the branching body lumen, (b) the wings are positioned in the main body lumen, and (c) the prosthesis transitions to the expanded state, such that (i) the wings come in contact with a wall of the main body lumen, (ii) the tube axis defines an angle of between 75 and 90 degrees with a central longitudinal axis of the main body lumen, (iii) the wings at least partially occupy respective arcs of the main body lumen, at least one of which arcs has an angle of no more than 180 degrees, and (iv) the wings have respective greatest axial lengths along the main body lumen axis, at least one of which is at least 1.5 times the diameter of the tubular structure.

For some applications, the main and branching body lumens are main and branching blood vessels, respectively. For some applications, the main and branching blood vessels are an aorta and a renal artery, respectively.

For some applications, the method further includes identifying the subject as suffering from the aortic aneurysm, and introducing includes transvascularly introducing the prosthesis responsively to the identifying.

For some applications, providing includes further providing a generally tubular self-expanding stent-graft, and further including deploying the stent-graft in the main body lumen within a space surrounded by the wings, such that the stent-graft expands to apply the one or more forces to the wings.

For some applications, the branching body lumen is one of a two or more branching body lumens that branch from the main body lumen, the prosthesis is one of a plurality of prostheses as in any of the applications described above, which include respective tubular structures a sets of wings, providing the prosthesis includes providing two or more of the prostheses, and deploying includes deploying the prostheses such that the tubular structures of the prostheses are positioned in respective ones of the branching body lumens, and the sets of wings of the prosthesis are positioned in the main body lumen.

For some applications, at least two of the branching body lumens branch from the main body lumen at different respective axial positions along the main body lumen, and providing and deploying the prostheses includes providing and deploying the prostheses so as to axially align respective proximal ends of at least two of the prostheses with one other.

For some applications, at least two of the branching body lumens branch from the main body lumen at different respective axial positions along the main body lumen, and providing and deploying the prostheses includes providing and deploying the prostheses so as to axially align respective distal ends of at least two of the prostheses with one other.

For some applications, providing two or more of the prostheses includes providing:

a first prosthesis, the wings of which together define a main portion of the first prosthesis, and the tubular structure of the first prosthesis joins the wings at a junction such that a center of the junction is positioned within 5% of an axial length of the main portion from an axial center of the main portion, and

a second prosthesis, the wings of which together define a main portion of the second prosthesis, and the tubular structure of the second prosthesis joins the wings at a junction such that a center of the junction is positioned greater than 10% of the axial length from the axial center of the main portion.

For some applications, providing the two or more prosthesis includes further providing a generally tubular self-expanding stent-graft, and further includes deploying the stent-graft in the main body lumen.

For some applications, providing the stent-graft includes providing the stent-graft in which no portion of the stent-graft is fixed to any of the prostheses.

There is still further provided, in accordance with an application of the present invention, a method including:

providing (a) two or more endovascular prostheses, which are configured to assume respective compressed states and expanded states, and which are shaped so as to define, when the prostheses assumes the expanded states, respective main portions and respective branching portions, which are coupled to the respective main portions, and (b) a generally tubular self-expanding stent-graft;

transvascularly introducing the prostheses into a main body lumen of a human subject, while the prostheses are positioned in one or more tubular delivery shafts in the compressed states;

deploying the prostheses from the one or more delivery shafts at a branch between the main body lumen and two or more branching body lumens, such that (a) the branching portions are positioned in respective ones of the branching body lumens, (b) the main portions are positioned in the main body lumen, and (c) the prostheses transition to the expanded states;

transvascularly introducing the stent-graft into the main body lumen, while the stent-graft is positioned in a delivery catheter in a compressed state; and

deploying the stent-graft from the delivery catheter in the main body lumen such that the stent-graft transitions to an expanded state and holds the main portions in place against a wall of the main body lumen.

For some applications, the main and branching body lumens are main and branching blood vessels, respectively. For some applications, the main and branching blood vessels are an aorta and two renal arteries, respectively.

For some applications, providing the prostheses includes providing the prostheses including respective fluid flow guides, which at least partially cover the respective main portions, and deploying the prostheses includes deploying the prostheses such that fluid flow guides form at least one fluid seal with each other.

For some applications, providing the prostheses includes providing the prostheses including respective prosthesis fluid flow guides, which at least partially cover the respective prostheses; providing the stent-graft includes providing the stent-graft including a stent-graft fluid flow guide, which covers at least a portion of the stent-graft; and deploying the stent-graft includes deploying the stent-graft such that the stent-graft fluid flow guide forms at least one fluid seal with the prosthesis fluid flow guides.

For some applications, the method further includes identifying the subject as suffering from the aortic aneurysm, and introducing includes transvascularly introducing the prostheses and the stent-graft responsively to the identifying.

For some applications, providing the stent-graft includes providing the stent-graft in which no portion of the stent-graft is fixed to any of the prostheses.

For some applications, providing the stent-graft includes providing the stent-graft including a stent-graft structural member and a blood-impervious stent-graft fluid flow guide attached to the stent-graft structural member, which fluid flow guide is shaped so as to define an axial discontinuation around at least a portion of a circumference of the stent-graft. For some applications, providing the stent-graft includes providing the stent-graft in which the stent-graft fluid flow guide is shaped so as to define the axial discontinuation entirely around the circumference of the stent-graft. For some applications, providing the stent-graft includes providing the stent-graft in which the axial discontinuation has a discontinuation length equal to between 50% and 85% of each of the shortest axial lengths of the wings, or between 60% and 70% of each of the shortest axial lengths of the wings.

The present invention will be more fully understood from the following detailed description of embodiments thereof, taken together with the drawings, in which:

### BRIEF DESCRIPTION OF THE DRAWINGS

FIGS. 1 and 2 are schematic illustrations of an endovascular prosthesis, in accordance with an application of the present invention;

FIG. 3 is a schematic illustration of the prosthesis of FIGS. 1 and 2 in a compressed state, initially positioned in a delivery shaft, in accordance with an application of the present invention;

FIG. 4 is a schematic illustration of the prosthesis of FIGS. 1 and 2 in the compressed state, in accordance with an application of the present invention;

FIG. 5 is a schematic illustration of the prosthesis of FIGS. 1 and 2 in an intermediate state of expansion, in accordance with an application of the present invention;

FIG. 6 is a schematic illustration of the prosthesis of FIGS. 1 and 2 deployed at a branch between an aneurysmatic abdominal aorta and a right renal artery, in accordance with an application of the present invention;

FIG. 7 is a schematic illustration of the deployment of two prostheses of FIGS. 1 and 2, in accordance with an application of the present invention;

FIG. 8 is a schematic illustration of the deployment of two prostheses of FIGS. 1 and 2, as well as a stent-graft, in accordance with an application of the present invention;

FIG. 9 is a schematic illustration of another configuration of the deployment of two prostheses of FIGS. 1 and 2 and a stent-graft, in accordance with an application of the present invention; and

FIG. 10 is a schematic illustration of yet another configuration of the deployment of two prostheses of FIGS. 1 and 2 and a stent-graft, in accordance with an application of the present invention.

### DETAILED DESCRIPTION OF APPLICATIONS

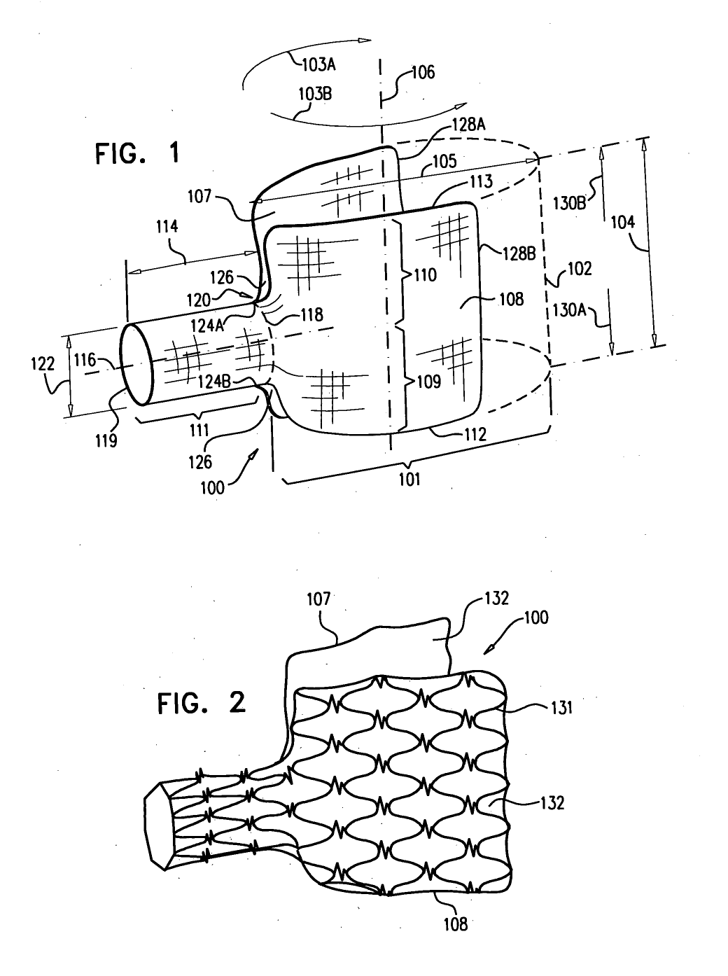

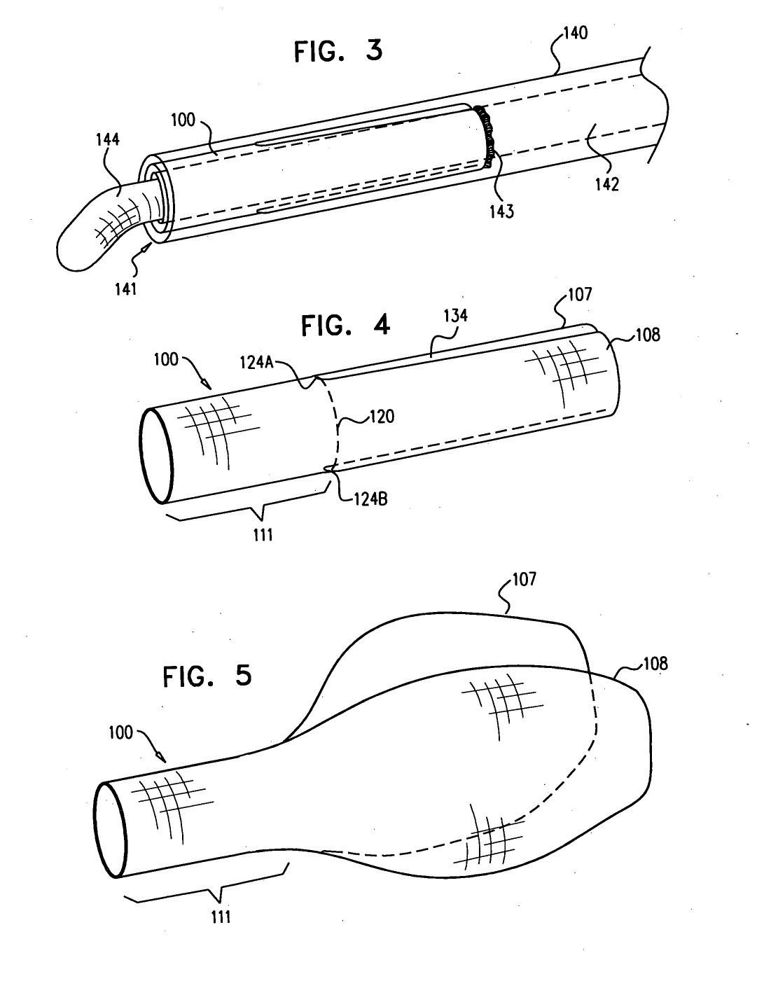

FIGS. 1 and 2 are schematic illustrations of an endovascular prosthesis 100, in accordance with an application of the present invention. Endovascular prosthesis 100 is configured to initially be positioned in a tubular delivery shaft (such as a catheter) in a compressed state, as described hereinbelow with reference to FIG. 3, and to assume an expanded state upon being deployed from the tubular delivery shaft, as described hereinbelow with reference to FIGS. 4-5. FIGS. 1 and 2 show the endovascular prosthesis in the expanded state. For some applications, the prosthesis is relaxed in the expanded state. For some applications, the prosthesis, and other prostheses and stent-grafts described herein, are configured to be self-expanding. For example, they'may be heat-set to assume the expanded state.

As shown in FIG. 2, prosthesis 100 comprises a structural member 131 and, optionally, a blood-impervious fluid flow guide 132. Structural member 131 typically comprises a plurality of structural stent elements. For some applications, at least some of, e.g., all of, the structural stent elements are interconnected (as shown in the figures), while for other applications, at least a portion of, e.g., all, of the structural stent elements are not interconnected (configuration not shown). For some applications, structural member 131 comprises a super-elastic alloy, such as Nitinol.

Prosthesis 100 is configured to be positioned at a branch between a main body lumen and a branching body lumen, such as a main blood vessel and a branching blood vessel. For example, the main body lumen may be an aorta, and the branching body lumen may be a renal artery.

When prosthesis 100 assumes its expanded state, respective portions of structural member 131 are shaped so as to define (a) a main portion 101, which is configured to be positioned in the main body lumen, and (b) a branching portion that comprises a substantially tubular structure 111, which is configured to be positioned in the branching body lumen.

For some applications, main portion 101 comprises two wings 107 and 108 (typically exactly two wings), which are coupled to a proximal end 118 of tubular structure 111 at generally opposite sides of the proximal end at a junction 120. The wings may be placed within and in contact with the main body lumen (or, alternatively, within and in contact with a right circular cylinder 102, which has a diameter 105 of between 2.5 and 3 cm). A distal end 119 of tubular structure 111 is thus positioned outside the main body lumen (or, more generally, outside the cylinder) in the branching body lumen. It is to be understood that the main and branching body lumens, as well as cylinder 102, are not elements of apparatus of the present invention, and are described, and recited in the claims, for purposes of helping to define the structure of the actual elements of the apparatus. Typically, when the prosthesis is thus positioned:
a central longitudinal axis 116 of tubular structure 111 defines an angle of between 75 and 90 degrees (such as between 85 and 90 degrees, e.g., 90 degrees) with a central longitudinal axis 106 of cylinder 102 or the main body lumen;
wings 107 and 108 at least partially occupy respective arcs 103A and 103B of cylinder 102 or the main body lumen, at least one of which arcs has an angle of no more than 180 degrees; and/or
wings 107 and 108 have respective greatest axial lengths 104 along axis 106, at least one of which is at least 1.5 times a diameter 122 of tubular structure 111, such as at least 2 times, or at least 2.5 times the diameter.

For some applications, the angle of the at least one of arcs 103A and 103B (such as of both of the arcs, taken separately) is at least 30 degrees, no more than 170 degrees, and/or between 30 and 170 degrees, such as at least 95 degrees, no more than 170 degrees, and/or between 95 and 170 degrees. Typically, each of the arcs 103A and 103B (separately) has an angle that is no more than 180 degrees. For some applications, a sum of the angles of the arcs 103A and 103B of wings 107 and 108 is less than 360 degrees, such as at least 90 degrees, no more than 270 degrees, and/or between 90 and 270 degrees. For some applications, respective lengths of arcs 103A and 103B are within 10% of each other, when prosthesis 100 assumes the expanded state and if the wings are placed within and in contact with the cylinder or the main body lumen. For some applications, at least one of arcs 103A and 103B (such as both of the arcs, taken separately) has a length of at least 2 cm, no more than 4 cm, and/or between 2 and 4 cm, such as 3 cm.

When prosthesis 100 assumes its expanded state, and if the wings are positioned within cylinder 102, when the prosthesis is viewed from one end of cylinder 102, one of the wings (e.g., wing 107) is positioned clockwise from junction 120, while the other wing (e.g., wing 108) is positioned counterclockwise from the junction.

For some applications, one or both of wings 107 and 108 define a proximal portion 109 that is positioned axially proximal to tube axis 116, and a distal portion 110 that is positioned axially distal to tube axis 116, when prosthesis 100 assumes the expanded state and if the wings are placed within and in contact with the cylinder or the main body lumen.

Main portion 101 is shaped so as to define a proximal end 112 and a distal end 113. For some applications, at least 50%, such as at least 80%, of proximal end 112 defines a line, and/or at least 50%, such as at least 80%, of proximal end 113 defines a line. For some applications, the lines defined by the proximal and distal ends 112 and 113 are generally parallel with each other.

For some applications, wings 107 and 108 are fixed to each other at exactly zero, exactly one, or exactly two points. For example, in the configuration shown in FIGS. 1 and 2, the wings are coupled to each other at exactly two points 124A and 124B, at generally opposite sides of proximal end 118 of tubular structure 111, at junction 120. For some applications, at least one of wings 107 and 108 (such as both wings) extends axially along the main body lumen or cylinder beyond proximal end 118 of tubular structure 111 in a first axial direction 130A and a second axial direction 130B opposite first direction 130A, when prosthesis 100 assumes the expanded state and if wings 107 and 108 are placed within and in contact with the cylinder or the main body lumen, such that the wings define a first gap 126 between the wings in first axial direction 130A, and a second gap 126 between the wings in second axial direction 130B.

For some applications, wings 107 and 108 are not fixed to each other at any points farther than 2 mm from proximal end 118 of tubular structure 111, when the prosthesis assumes the expanded state and if the wings are placed within and in contact with the cylinder or the main body lumen.

Alternatively or additionally, for some applications, wings 107 and 108 are shaped so as to define at least one gap 126 therebetween (e.g., exactly two gaps 126) near proximal end 118 of tubular structure 111 (e.g., such that at least respective portions of each of gaps 126 is within 1 cm of the proximal end).

For some applications, prosthesis 100 is configured such that if wings 107 and 108 are placed within the main body lumen or cylinder 102, the wings contact the main body lumen or the cylinder even if no radial forces are applied to the wings. Thus, for example, in its expanded state the prosthesis may assume the shape shown in FIGS. 1 and 2, if placed on a flat surface without application of any radial forces, even if not placed within the main body lumen or cylinder.

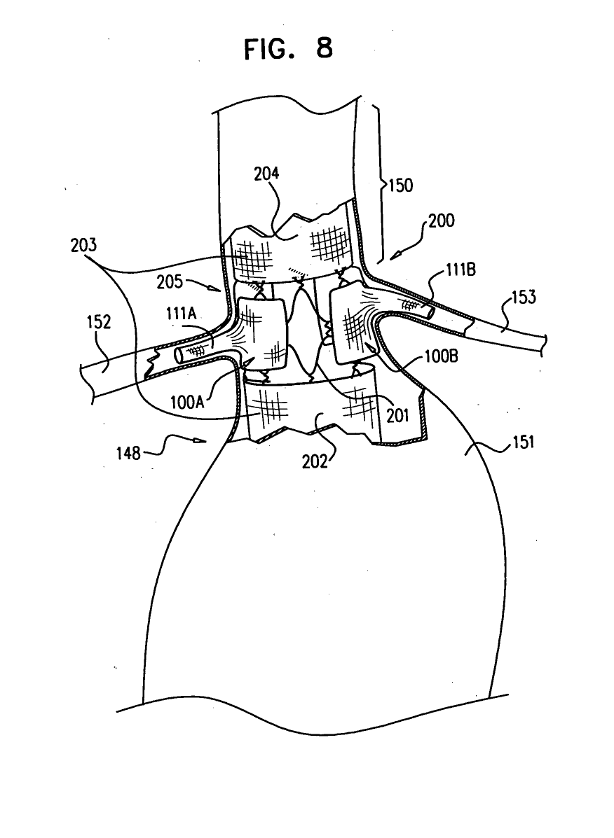

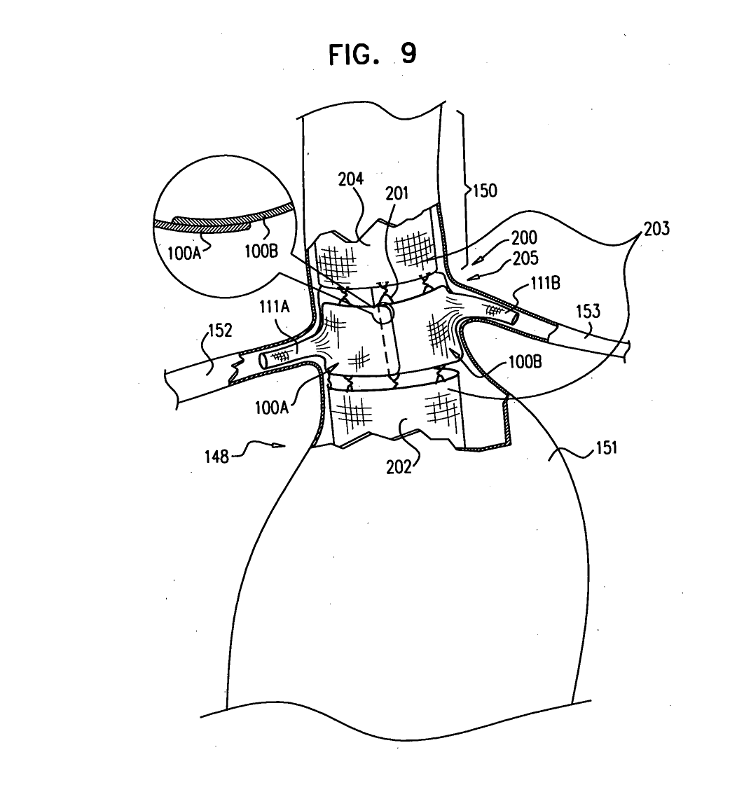

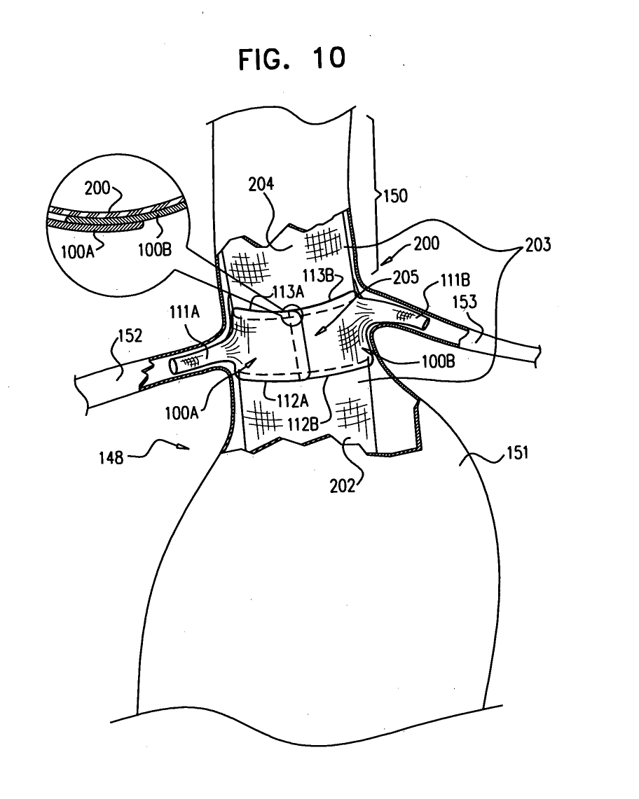

For other applications, prosthesis 100 is configured such that if wings 107 and 108 are placed within the main body lumen or the cylinder, the wings contact the main body lumen or cylinder only if one or more forces are applied to the wings in one or more radially-outward directions. In the absence of application of such forces, one or both of the wings would be positioned closer to axis 106 of the main body lumen or cylinder. For some applications, the radially-outward forces are applied by stent-graft 200, such as described hereinbelow with reference to FIGS. 8, 9, and/or 10. Stent-graft 200 is configured and sized to apply the one or more forces to the wings upon expansion of the stent-graft.

For still other applications, prosthesis 100 is configured such that if the wings are placed within the main body lumen or the cylinder, the wings contact the cylinder only if one or more forces are applied radially inwardly on the wings by the main body lumen or the cylinder. In the absence of application of such forces, one or both of the wings would be positioned outside cylinder 102. As the prosthesis transitions from its initial contracted state towards a maximum possible expansion, the expansion is constrained by the wall of the main body lumen or the inner surface of the cylinder, causing the wings to assume the shape shown in FIGS. 1 and 2.

For some applications, at least one of wings 107 and 108 (such as both wings) extends axially along the main body lumen or cylinder beyond proximal end 118 of tubular structure 111 in first axial direction 130A and second axial direction 130B opposite first direction 130A, when prosthesis 100 assumes the expanded state and if wings 107 and 108 are placed within and in contact with the cylinder or the main body lumen.

Prosthesis 100 may be configured such that tubular structure 111 joins wings 107 and 108 at various axial positions along wings 107 and 108 (and main portion 101). For some applications, such as described hereinbelow with reference to FIG. 10, a plurality of prostheses 100 may be provided in which the tubular structure joins the wings at respective differing axial positions. For some applications, the axial positions provided include at least:
an axial position at or near an axial center of the wings; for example, tubular structure 111 may join the wings such that a center of junction 120 is positioned within 5% of an axial length of main portion 101 from an axial center of main portion 101 (as a result, proximal and distal portions 109 and 110 have approximately the same axial lengths); and/or
an axial position offset from the axial center of the wings; for example, tubular structure 111 may join the wings such that a center of junction 120 is positioned greater than 10% of an axial length of main portion 101 from the axial center of main portion 101, such as greater than 25% (as a result, proximal and distal portions 109 and 110 have different axial lengths).

For some applications, respective tube-coupling portions of wings 107 and 108 are coupled to proximal end 118 of tubular structure 111. Wings 107 and 108 are shaped so as to define respective end portions 128A and 128B farthest from the tube-coupling portions, when the prosthesis assumes the expanded state and if the wings are placed within and in contact with the cylinder or the main body lumen. End portions 128A and 128B are not fixed to each other. For some applications, one or both of end portions 128A and 128B are generally perpendicular to proximal end 112 and/or a distal end 113.

Typically, tubular structure 111 has an axial length 114 of at least 1 cm, no more than 5 cm, and/or between 1 and 5 cm, such as 2 cm, and a diameter of at least 3 mm, no more than 12 mm (e.g., no more than 8 mm), and/or between 3 and 12 mm (e.g., between 3 and 8 mm), such as 8 mm. Tubular structure 111 defines a central longitudinal axis 116.

For applications in which fluid flow guide 132 is provided, the fluid flow guide typically comprises at least one biologically-compatible substantially fluid-impervious flexible sheet, which is attached (such as by stitching) to at least a portion of structural member 131, on either side of the surfaces defined by the structural member. For some applications, fluid flow guide 132 is attached to structural member 131 such that the fluid flow guide entirely covers both of wings 107 and 108, such that the fluid flow guide creates a blood-impervious continuum together with the wings. Alternatively, the fluid flow guide is attached to the structural member such that the fluid flow guide only partially covers each of the wings. For example, the fluid flow guide may cover only proximal portions 109 of the wings, or a portion (e.g., a proximal portion) of proximal portions 109. For some applications, the fluid flow guide is attached to the structural member such that the fluid flow guide covers (either an external or an internal surface of) at least a portion of tubular structure 111, which portion extends from proximal end 118 of the tubular structure toward distal end 119 of the tubular structure. Optionally, the fluid flow guide covers the entire tubular structure. The flexible sheet may comprise, for example, a polymeric material (e.g., polytetrafluoroethylene), a textile material (e.g., polyethylene terephthalate (PET)), natural tissue (e.g., saphenous vein or collagen), or a combination thereof.

Reference is made to FIGS. 3 and 4, which are schematic illustrations of prosthesis 100 in its compressed state, in accordance with an application of the present invention. FIG. 3 shows the prosthesis in its compressed state initially positioned in an elongated tubular delivery shaft 140, disposed at a distal end 141 of the shaft. FIG. 4 shows the prosthesis in its compressed state, while, for clarity of illustration, not showing delivery shaft 140. Typically, prosthesis 100 is initially positioned within shaft 140 such that the prosthesis is entirely within the shaft. Typically, delivery shaft 140 is configured to slidably release prosthesis 100 therefrom during an implantation procedure.

For some applications, delivery shaft 140 further comprises an elongated inner shaft 142, which is positioned within the delivery shaft, and which is configured to prevent movement of prosthesis 100 in a distal direction within the delivery shaft. For example, for preventing such distal movement, inner shaft 142 may comprise a circumferential stopper 143 that is located distally adjacent to prosthesis 100. For some applications, inner shaft 142 is shaped so as to define an inner lumen (e.g., concentric), which is configured to allow insertion of a guidewire 144 therethrough.

For some applications, prosthesis 100 is configured to be positioned in delivery shaft 140 in its compressed state such that:
tube axis 116 coincides with a central longitudinal axis of delivery shaft 140;
tubular structure 111 is radially compressed around tube axis 116, for example to a compressed external diameter of at least 1 mm, no more than 3 mm, and/or between 1 and 3 mm; the axial length of tubular structure 111 when compressed may be, for example, at least 0.5 cm, no more than 4 cm, and/or between 0.5 and 4 cm;
wings 107 and 108 curve around the axis of the delivery shaft and subtend respective arcs of the delivery shaft; wings 107 and 108 may thus together define at least a portion of (e.g., at least 150 degrees), such as all of, a generally tubular shape, for example having a diameter of at least 1.5 mm, no more than 4 mm, and/or between 1.5 mm and 4 mm; the axial length of the wings when compressed may be, for example, at least 0.5 cm, no more than 4 cm, and/or between 0.5 and 4 cm;
wings 107 and 108 are aligned alongside each other (such as generally parallel to each other), and (whether or not the wings overlap) define therebetween two slits 134, which typically extend along the entire length of compressed prosthesis 100 other than tubular structure 111; for example, an axial length of each of the slits may be equal to at least 20%, no more than 80%, and/or between 20% and 80% of a total length of the compressed prosthesis (it is noted that the portion of the compressed prosthesis that defines the wings and the slits expands to a greater extent than the portion of the compressed prosthesis that defines the tubular structure, when the prosthesis transitions from the compressed state to the expanded state); and/or
tubular structure 111, wing 107, and wing 108 together define at least a portion of (e.g., at least 70 degrees), such as all of, a generally tubular shape.

For some applications, prosthesis 100 is configured to be positioned in delivery shaft 140 in the compressed state such that the subtended arcs of the delivery shaft do not overlap each other. For example, a sum of the non-overlapping subtended arcs of the delivery shaft may be at least 350 degrees. For other applications, prosthesis 100 is configured to be positioned in delivery shaft 140 in the compressed state such that the subtended arcs of the delivery shaft overlap each other.

Reference is made to FIG. 5, which is a schematic illustration of prosthesis 100 in an intermediate state of expansion, in accordance with an application of the present invention. This intermediate state is between the prosthesis's compressed state, as shown in FIGS. 3 and 4, and its expanded state, as shown in FIGS. 1 and 2. The prosthesis may assume this intermediate state for an instant during expansion after the prosthesis is deployed from delivery shaft 140.

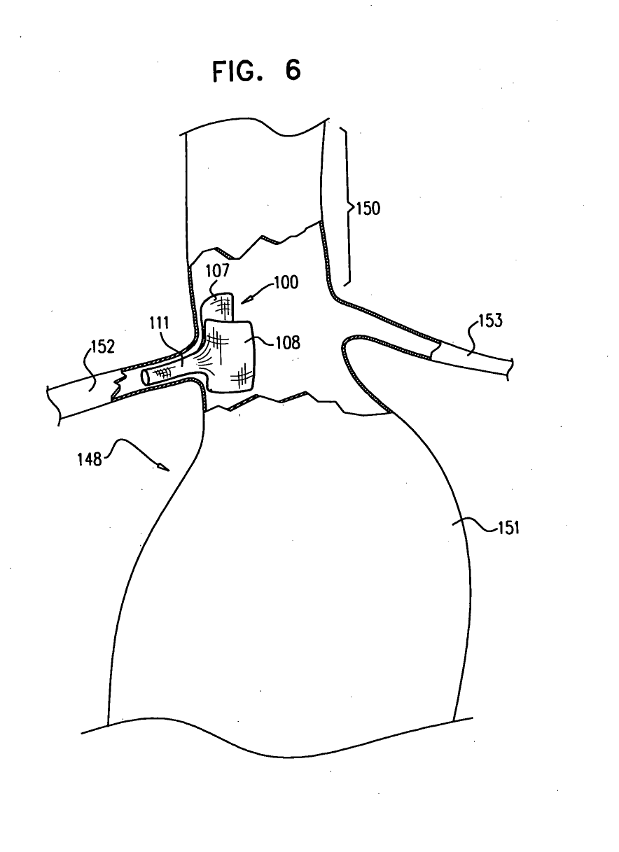

Reference is made to FIG. 6, which is a schematic illustration of prosthesis 100 deployed at a branch between an aneurysmatic abdominal aorta 148 and a right renal artery 152, in accordance with an application of the present invention. Aneurysmatic abdominal aorta 148 is shown including a supra-renal aorta 150 and an abdominal aneurysm 151. (Although not shown in FIG. 6, prosthesis 100 may of course instead be deployed in a left renal artery 153.) Tubular structure 111 is positioned within right renal artery 152, while wings 107 and 108 are positioned within the aorta, such that a first portion of each of the wings is rostral (proximal) to the renal artery, and a second portion is caudal (distal) to the renal artery. It is noted that the diameter of the aorta in the vicinity of the renal arteries is typically between 2.5 and 3 cm in adult humans. Typically, end portions 128A and 128B of wings 107 and 108 (FIG. 1) do not touch each other when prosthesis 100 is placed in the aorta.

For some applications, prosthesis 100 is deployed using delivery shaft 140, described hereinabove with reference to FIG. 3. For some applications, in order to implant prosthesis 100, the prosthesis is transvascularly (typically percutaneously) introduced into the aorta via one of the iliac arteries, while the prosthesis is positioned in delivery shaft 140 in the compressed state. Alternatively, for some applications, the prosthesis is instead deployed via a subclavian artery. Delivery shaft 140 are advanced over guidewire 144 until the guidewire is positioned in one of the renal arteries. The guidewire is withdrawn, leaving delivery shaft 140 in place, partially in the renal artery, and partially in the aorta. Inner shaft 142 is held in place as delivery shaft 140 is withdrawn, thereby delivering prosthesis 100 from the delivery shaft. Prosthesis 100 typically self-expands, thereby completing the delivery procedure. Alternatively, the prosthesis is delivered using an over-the-wire (OTW) approach, in which the guidewire is left in place until the prosthesis is expanded, and thereafter the guidewire is withdrawn.

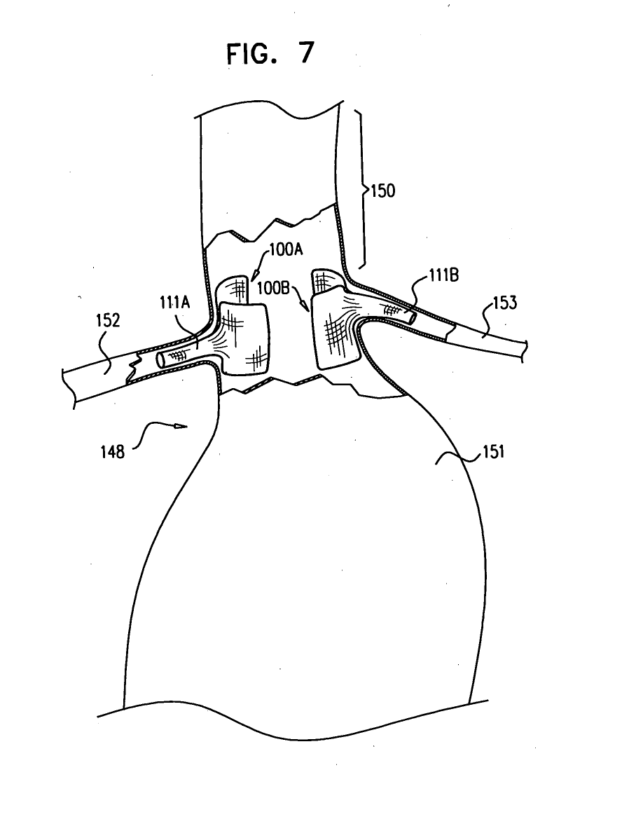

Reference is made to FIG. 7, which is a schematic illustration of the deployment of two prostheses 100A and 100B, in accordance with an application of the present invention. Prosthesis 100A is deployed at the branch between aorta 148 and right renal artery 152, while prosthesis 100B is deployed at a branch between aorta 148 and left renal artery 153. Typically, each of the prostheses is deployed using a separate delivery shaft 140, by reusing the safe delivery shaft to separately deliver each of the prostheses, or by initially loading both prostheses into a single delivery shaft, and delivering them one after the other. Typically, end portions 128A and 128B of wings 107 and 108 (FIG. 1) of each of the prostheses do not touch each other when prosthesis 100 is placed in the aorta. This may provide a gap through which the tubular structure of the other prosthesis may pass into the other renal artery.

Reference is made to FIG. 8, which is a schematic illustration of the deployment of two prostheses 100A and 100B, as well as a stent-graft 200, in accordance with an application of the present invention. Prostheses 100A and 100B are deployed as described with reference to FIG. 7. In addition, stent-graft 200 is deployed, which may, for example, be generally tubular and/or self-expanding, and is configured to be positioned in the main body lumen, such as the aorta. For some applications, stent-graft 200 implements all or a portion of the techniques described in one or more of the applications incorporated hereinbelow, mutatis mutandis. For some applications, stent-graft 200 has an expanded (e.g., relaxed) diameter of between 100 to 120 percent of the diameter of supra-renal aorta 150 (and/or cylinder 102). For some applications, stent-graft 200 is bifurcated at its caudal end (which is proximal to the surgeon, and distal to the heart), such as described in one or more of the applications incorporated hereinbelow by reference.

For some applications, as shown in FIG. 8, stent-graft 200 comprises a stent-graft structural member 201 and a blood-impervious stent-graft fluid flow guide 203 attached to the stent-graft structural member. For some applications, the fluid flow guide is shaped so as to define an axial discontinuation 205 around at least a portion of a circumference of the stent-graft, such entirely around the circumference, such that fluid flow guide 203 defines first and second portions 202 and 204. For example, first portion 204 may be positioned in supra-renal aorta 150, and second portion 202 may be positioned in the aorta below the renal arteries. Axial discontinuation 295 typically axially overlaps with the lengths of the endovascular prostheses 100A and 100B. For some applications, axial discontinuation 295 has a discontinuation length equal to between 50% and 85% of each of the shortest axial lengths of wings 207 and 208, such as between 60% and 70%.

Reference is made to FIG. 9, which is a schematic illustration of another configuration of the deployment of two prostheses 100A and 100B and stent-graft 200, in accordance with an application of the present invention. This configuration is similar to the configuration described hereinabove with reference to FIG. 8, except that prostheses 100A and 100B are sized to circumferentially overlap with each other, typically at two sites (only one of the overlap sites is visible in FIG. 9; the other site is along the opposite side not visible in the figure). This overlap serves to provide a fluid seal, thereby defining a fluid flow path through the four wings of the two prostheses. Such an overlap may be particularly appropriate for patients who lack an Endovascular Aneurysm Repair (EVAR)-suitable sub-renal neck. Typically, the sum of the angles of arcs 103A and 103B of prosthesis 100A and arcs 103A and 103B of prosthesis 100B is greater than 360 degrees, such as at least 400 degrees. In this configuration, wings 107 and 108 of both prostheses 100A and 100B are typically entirely covered by respective fluid flow guides 132 of the prostheses.

Reference is made to FIG. 10, which is a schematic illustration of yet another configuration of the deployment of two prostheses 100A and 100B and stent-graft 200, in accordance with an application of the present invention. This configuration is similar to the configuration described hereinabove with reference to FIG. 9, except that axial discontinuation 205 of stent-graft 200 has an axial length that is less than greatest axial lengths 104 of all of wings 107 and 108 of both prostheses 100A and 100B. As a result, the fluid flow guide covering first and second portions 202 and 204 of stent-graft 200 axially overlap with the fluid flow guides covering the wings, thereby providing a fluid flow path from second portion 204, through the wings, and into first portion 202.

For some applications, stent-graft 200 comprises second portion 204 (e.g., configured to be positioned below the renal arteries), but not first portion 202 (configuration not shown). Second portion may or may not be configured to axially overlap with the wings. For applications in which such axial overlap is provided, a fluid flow path is provided through the wings and into second portion 204.

In some applications of the present invention, a kit is provided that comprises two or more prostheses 100, such as exactly two prostheses 100. For some applications, the kit further comprises stent-graft 200, which may or may not define axial discontinuation 205. Typically, no portion of stent-graft 200 is fixed to any of prostheses 100. For some applications, the elements of the kit are deployed by first deploying and positioning a first one of prostheses 100 in one of the renal arteries, then deploying and positioning a second one of prostheses 100 in the other of the renal arteries, and subsequently deploying stent-graft 200 within the wings of the two prostheses 100. Radial expansion of stent-graft 200 within the wings holds prostheses 100 in place against a wall of the aorta (or other main body lumen).

Because prostheses 100 are separately deployed, each can be properly positioned in one of the renal arteries, even though the renal arteries generally branch from the aorta at different respective axial positions along the aorta. In order to make a provide a consistent overlap and fluid-tight connection between fluid flow guide 203 of stent-graft 200 and fluid flow guides 132 of prostheses 100, it is generally desirable to position the prostheses such that proximal (e.g., caudal) ends 112A and 112B of prostheses 100A and 100B are axially aligned with each other, and/or distal (e.g., rostral) ends 113A and 113B of prostheses 100A and 100B are axially aligned with each other.

In order to facilitate such alignment, for some applications the kit includes a plurality of prostheses 100 in which tubular structure 111 joins wings 107 and 108 at respective differing axial positions along main portion 101. The surgeon selects two of the prostheses with appropriately-positioned tubular structures, to provide the desired axial alignment of the proximal and/or distal ends. For some applications, the desired positioning may alternatively or additionally be achieved by selecting an axial orientation of the prosthesis. For example, in the exemplary configuration shown in FIG. 10, tubular structures 111A and 111B may join prostheses 100A and 100B at the same axial positions, except that prosthesis 100B has been axially inverted with respect to prosthesis 100A.

For some applications, prosthesis 100 comprises one or more anchoring elements that extend radially outwardly when the prosthesis assumes the expanded state. The anchoring elements anchor the prosthesis to a vascular wall, helping prevent dislodgement.

In the present application, including in the claims, the term “rostral” means closer to the heart via the aortic vasculature, and the term “caudal” means further from the heart via the aortic vasculature. For example, the renal arteries are “rostral” to the aorto-iliac bifurcation.

Although prostheses 100 and stent-graft 200 have sometimes been described hereinabove as being deployed at the branch of one or more renal arteries from the aorta, the prostheses and stent-graft may, for some applications, also be deployed at other branching body lumens. For example:
the main body lumen may be the aorta, and the branching body lumen may include the inferior or superior mesenteric arteries, or the celiac artery; when sized for these branches, prosthesis 100 may have some or all of the properties described hereinabove if placed in cylinder 102 having a diameter of between 2.5 and 5.5 cm;
the main body lumen may be the aorta and the branching body lumens may include both iliac arteries; when sized for these branches, prosthesis 100 may have some or all of the properties described hereinabove if placed in cylinder 102 having a diameter of between 2 and 5.5 cm; or
the main body lumen may be the aortic arch and the branching body lumen may include the brachiocephalic artery, the left common carotid artery, and/or the subclavian artery; when sized for these branches, prosthesis 100 may have some or all of the properties described hereinabove if placed in cylinder 102 having a diameter of between 3 and 8 cm.

The scope of the present invention includes embodiments described in the following applications, which are assigned to the assignee of the present application and are incorporated herein by reference. In an embodiment, techniques and apparatus described in one or more of the following applications are combined with techniques and apparatus described herein:
PCT Application PCT/IL2008/000287, filed Mar. 5, 2008, which published as PCT Publication WO 2008/107885 to Shalev et al.
U.S. application Ser. No. 12/529,936, which published as US Patent Application Publication 2010/0063575 to Shalev et al.
US Provisional Application 60/892,885, filed Mar. 5, 2007
US Provisional Application 60/991,726, filed Dec. 2, 2007
US Provisional Application 61/219,758, filed Jun. 23, 2009
US Provisional Application 61/221,074, filed Jun. 28, 2009
PCT Application PCT/IB2010/052861, filed Jun. 23, 2010
a PCT application filed Jul. 14, 2010, entitled, “Sideport engagement and sealing mechanism for endoluminal stent-grafts”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a tubular delivery shaft, the apparatus comprising an endovascular prosthesis, which is configured to initially be positioned in the delivery shaft in a compressed state, and to assume an expanded state upon being deployed from the delivery shaft, and which comprises a structural member, respective portions of which are shaped so as to define, when the prosthesis assumes the expanded state:
a substantially tubular structure, having proximal and distal ends, a diameter, and a central longitudinal tube axis, and
two wings, which are coupled to the proximal end of the tubular structure at generally opposite sides of the proximal end, such that, if the wings are placed within and in contact with a right circular cylinder, which has a diameter of between 2.5 and 3 cm, such that the distal end of the tubular structure is outside the cylinder:
the tube axis defines an angle of between 75 and 90 degrees with a central longitudinal axis of the cylinder,
the wings at least partially occupy respective arcs of the cylinder, at least one of which arcs has an angle of no more than 180 degrees, and
the wings have respective greatest axial lengths along the cylinder axis, at least one of which is at least 1.5 times the diameter of the tubular structure.

2. (canceled)

3. The apparatus according to claim 1, wherein the angle of the at least one of the arcs is between 95 and 170 degrees.

4-7. (canceled)

8. The apparatus according to claim 1, wherein the two wings are fixed to each other and are not fixed to each other at any points farther than 2 mm from the proximal end of the tubular structure, when the prosthesis assumes the expanded state and if the wings are placed within and in contact with the cylinder.

9-10. (canceled)

11. The apparatus according to claim 1, wherein the two wings are fixed to each other at, exactly one or exactly two points.

12. The apparatus according to claim 11, wherein, when the prosthesis assumes the expanded state and if the wings are placed within and in contact with the cylinder, each of the wings extends axially along the cylinder beyond the proximal end of the tubular structure in a first axial direction and a second axial direction opposite the first direction, such that the wings define a first gap between the wings in the first axial direction, and a second gap between the wings in the second axial direction.

13. (canceled)

14. The apparatus according to claim 1, wherein the prosthesis is configured such that if the wings are placed within the cylinder, the wings contact the cylinder only if one or more forces are applied to the wings in one or more radially-outward directions.

15. The apparatus according to claim 14, further comprising, in addition to and separate from the endovascular prosthesis, a generally tubular self-expanding stent-graft, which (a) comprises a stent-graft structural member and a blood-impervious stent-graft fluid flow guide attached to the stent-graft structural member, and (b) is configured and sized to apply the one or more forces to the wings upon expansion of the stent-graft.

16. (canceled)

17. The apparatus according to claim 1,
wherein respective tube-coupling portions of the wings are coupled to the proximal end of the tubular structure,
wherein the wings are shaped so as to define respective end portions farthest from the tube-coupling portions, when the prosthesis assumes the expanded state and if the wings are placed within and in contact with the cylinder, and
wherein the end portions are not fixed to each other.

18-20. (canceled)

21. The apparatus according to claim 1, wherein the prosthesis is configured to be positioned in the delivery shaft in the compressed state such that:
the tube axis of the tubular structure coincides with a central longitudinal axis of the delivery shaft, and
the wings curve around the axis of the delivery shaft and subtend respective arcs of the delivery shaft.

22. The apparatus according to claim 21, wherein the two wings together define at least a portion of a generally tubular shape, when the prosthesis is positioned within the delivery shaft.

23. The apparatus according to claim 21, wherein the two wings and the tubular structure together define at least a portion of a generally tubular shape, when the prosthesis is positioned within the delivery shaft.

24-28. (canceled)

29. The apparatus according to claim 1, wherein the prosthesis further comprises a blood-impervious fluid flow guide, which is attached to at least a portion of the structural member, and wherein the fluid flow guide is attached to the structural member such that the fluid flow guide entirely covers both of the wings, such that the fluid flow guide creates a blood-impervious continuum together with the wings.

30. The apparatus according to claim 1, wherein the prosthesis further comprises a blood-impervious fluid flow guide, which is attached to at least a portion of the structural member, and wherein the fluid flow guide is attached to the structural member such that the fluid flow guide only partially covers each of the wings.

31-43. (canceled)

44. The apparatus according to claim 1, wherein the prosthesis is configured to be positioned at a branch between a main body lumen and a branching body lumen, such that the tubular structure is positioned within the branching body lumen, and the wings are positioned within the main body lumen.

45. The apparatus according to claim 44, wherein the main body lumen is an aorta, and the branching body lumen is a renal artery, and the prosthesis is configured to be positioned such that the tubular structure is positioned within the renal artery, and the wings are positioned within the aorta.

46. The apparatus according to claim 1, wherein the prosthesis is one of plurality of prostheses as recited in claim 1, and wherein the apparatus comprises a kit, which comprises two or more of the prostheses.

47-48. (canceled)

49. The apparatus according to claim 46, wherein the kit further comprises, in addition to and separate from the prostheses, a generally tubular self-expanding stent-graft, which comprises a stent-graft structural member and a blood-impervious stent-graft fluid flow guide attached to the stent-graft structural member.

50. (canceled)

51. The apparatus according to claim 49, wherein the fluid flow guide is shaped so as to define an axial discontinuation around at least a portion of a circumference of the stent-graft.

52-54. (canceled)

55. A method comprising:
providing an endovascular prosthesis, which is configured to assume a compressed state and an expanded state, and which includes a structural member, respective portions of which are shaped so as to define, when the prosthesis assumes the expanded state: (a) a substantially tubular structure, having proximal and distal ends, a diameter, and a central longitudinal tube axis, and (b) two wings, which are coupled to the proximal end of the tubular structure at generally opposite sides of the proximal end;
transvascularly introducing the prosthesis into a main body lumen of a human subject, while the prosthesis is positioned in a tubular delivery shaft in the compressed state; and
deploying the prosthesis from the delivery shaft at a branch between the main body lumen and a branching body lumen, such that (a) the tubular structure is positioned in the branching body lumen, (b) the wings are positioned in the main body lumen, and (c) the prosthesis transitions to the expanded state, such that (i) the wings come in contact with a wall of the main body lumen, (ii) the tube axis defines an angle of between 75 and 90 degrees with a central longitudinal axis of the main body lumen, (iii) the wings at least partially occupy respective arcs of the main body lumen, at least one of which arcs has an angle of no more than 180 degrees, and (iv) the wings have respective greatest axial lengths along the main body lumen axis, at least one of which is at least 1.5 times the diameter of the tubular structure.

56-57. (canceled)

58. The method according to claim 55, further comprising identifying the subject as suffering from the aortic aneurysm, wherein introducing comprises transvascularly introducing the prosthesis responsively to the identifying.

59. (canceled)

60. The method according to claim 55, wherein providing comprises providing the prosthesis in which the angle of the at least one of the arcs is between 95 and 170 degrees.

61-64. (canceled)

65. The method according to claim 55, wherein providing comprises providing the prosthesis in which the two wings are fixed to each other and are not fixed to each other at any points farther than 2 mm from the proximal end of the tubular structure, when the prosthesis assumes the expanded state and the wings are placed within and in contact with the main body lumen.

66-67. (canceled)

68. The method according to claim 55, wherein providing comprises providing the prosthesis in which the two wings are fixed to each other at, exactly one or exactly two points.

69-70. (canceled)

71. The method according to claim 55, wherein the prosthesis is configured such that when the wings are placed within the main body lumen, the wings contact the main body lumen only if one or more forces are applied to the wings in one or more radially-outward directions.

72. The method according to claim 71, wherein providing comprises further providing, in addition to and separate from the endovascular prosthesis, a generally tubular self-expanding stent-graft, which comprises a stent-graft structural member and a blood-impervious stent-graft fluid flow guide attached to the stent-graft structural member, and further comprising deploying the stent-graft in the main body lumen within a space surrounded by the wings, such that the stent-graft expands to apply the one or more forces to the wings.

73. (canceled)

74. The method according to claim 55, providing comprises providing the prosthesis in which respective tube-coupling portions of the wings are coupled to the proximal end of the tubular structure, the wings are shaped so as to define respective end portions farthest from the tube-coupling portions, when the prosthesis assumes the expanded state and the wings are placed within and in contact with the main body lumen, and the end portions are not fixed to each other.

75-77. (canceled)

78. The method according to claim 55, wherein transvascularly introducing comprises transvascularly introducing the prosthesis while the prosthesis is initially positioned in the delivery shaft in the compressed state such that:
the tube axis of the tubular structure coincides with a central longitudinal axis of the delivery shaft, and
the wings curve around the axis of the delivery shaft and subtend respective arcs of the delivery shaft.

79. The method according to claim 78, wherein the two wings together define at least a portion of a generally tubular shape, when the prosthesis is positioned within the delivery shaft.

80. The method according to claim 78, wherein the two wings and the tubular structure together define at least a portion of a generally tubular shape, when the prosthesis is positioned within the delivery shaft.

81-85. (canceled)

86. The method according to claim 55, wherein providing comprises providing the prosthesis in which the prosthesis further includes a blood-impervious fluid flow guide, which is attached to at least a portion of the structural member, which fluid flow guide is attached to the structural member such that the fluid flow guide entirely covers both of the wings, such that the fluid flow guide creates a blood-impervious continuum together with the wings.

87. The method according to claim 55, wherein providing comprises providing the prosthesis in which the prosthesis further includes a blood-impervious fluid flow guide, which is attached to at least a portion of the structural member, which fluid flow guide is attached to the structural member such that the fluid flow guide only partially covers each of the wings.

88-99. (canceled)

100. The method according to claim 55,
wherein the branching body lumen is one of a two or more branching body lumens that branch from the main body lumen,
wherein the prosthesis is one of a plurality of prostheses as recited in claim 55, which include respective tubular structures a sets of wings,
wherein providing the prosthesis comprises providing two or more of the prostheses, and
wherein deploying comprises deploying the prostheses such that the tubular structures of the prostheses are positioned in respective ones of the branching body lumens, and the sets of wings of the prosthesis are positioned in the main body lumen.

101-104. (canceled)

105. The method according to claim 100, wherein providing the two or more prosthesis comprises further providing, in addition to and separate from the prostheses, a generally tubular self-expanding stent-graft, which comprises a stent-graft structural member and a blood-impervious stent-graft fluid flow guide attached to the stent-graft structural member, and further comprises deploying the stent-graft in the main body lumen.

106. (canceled)

107. The method according to claim 105, wherein providing the stent-graft comprises providing the stent-graft in which the fluid flow guide is shaped so as to define an axial discontinuation around at least a portion of a circumference of the stent-graft.

108-110. (canceled)

111. A method comprising:
providing (a) two or more endovascular prostheses, which are configured to assume respective compressed states and expanded states, and which are shaped so as to define, when the prostheses assumes the expanded states, respective main portions and respective branching portions, which are coupled to the respective main portions, and (b) a generally tubular self-expanding stent-graft;
transvascularly introducing the prostheses into a main body lumen of a human subject, while the prostheses are positioned in one or more tubular delivery shafts in the compressed states;
deploying the prostheses from the one or more delivery shafts at a branch between the main body lumen and two or more branching body lumens, such that (a) the branching portions are positioned in respective ones of the branching body lumens, (b) the main portions are positioned in the main body lumen, and (c) the prostheses transition to the expanded states;
transvascularly introducing the stent-graft into the main body lumen, while the stent-graft is positioned in a delivery catheter in a compressed state; and
deploying the stent-graft from the delivery catheter in the main body lumen such that the stent-graft transitions to an expanded state and holds the main portions in place against a wall of the main body lumen.

112-113. (canceled)

114. The method according to claim 111,
wherein providing the prostheses comprises providing the prostheses including respective fluid flow guides, which at least partially cover the respective main portions, and
wherein deploying the prostheses comprises deploying the prostheses such that fluid flow guides form at least one fluid seal with each other.

115. The method according to claim 111,
wherein providing the prostheses comprises providing the prostheses including respective prosthesis fluid flow guides, which at least partially cover the respective prostheses,
wherein providing the stent-graft comprises providing the stent-graft including a stent-graft fluid flow guide, which covers at least a portion of the stent-graft, and
wherein deploying the stent-graft comprises deploying the stent-graft such that the stent-graft fluid flow guide forms at least one fluid seal with the prosthesis fluid flow guides.

116. The method according to claim 111, further comprising identifying the subject as suffering from the aortic aneurysm, wherein introducing comprises transvascularly introducing the prostheses and the stent-graft responsively to the identifying.

117. The method according to claim 111, wherein providing the stent-graft comprises providing the stent-graft in which no portion of the stent-graft is fixed to any of the prostheses.

118-121. (canceled)

122. The apparatus according to claim 1, wherein the two wings collectively are coupled to an entire perimeter of the proximal end of the tubular structure.

123. The method according to claim 55, wherein providing comprises providing the prosthesis in which the two wings collectively are coupled to an entire perimeter of the proximal end of the tubular structure.
